(19) United States
(12) Patent Application Publication
(10) Pub. No.: US 2017/0354617 A1
(43) Pub. Date: Dec. 14, 2017

(54) METHOD OF TREATING OR PREVENTING OSTEOPOROSIS

(71) Applicant: Research Foundation of the City University of New York, New York, NY (US)

(72) Inventors: Dorra Frikha-Benayed, Clifton, NJ (US); Mitchell B. Schaffler, Rye, NY (US)

(21) Appl. No.: 15/618,783

(22) Filed: Jun. 9, 2017

Related U.S. Application Data
(60) Provisional application No. 62/347,745, filed on Jun. 9, 2016.

(51) Int. Cl. A61K 31/12; A61K 38/17; A61K 31/00; G01N 33/15

(52) U.S. Cl. CPC A61K 31/12; A61K 31/00; A61K 38/17; G01N 33/15

(57) ABSTRACT

A method for treating or preventing osteoporosis is provided that treats a patient with methylene blue.

### CROSS-REFERENCE TO RELATED APPLICATIONS

This application claims priority to and is a non-provisional of U.S. Patent Application 62/347,745 (filed Jun. 9, 2016), the entirety of which is incorporated herein by reference.

### STATEMENT OF FEDERALLY SPONSORED RESEARCH OR DEVELOPMENT

This invention was made with government support under grant number AR041210 awarded by National Institute of Health (NIH). The government has certain rights in the invention.

### BACKGROUND OF THE INVENTION

Osteoporosis is a common disease characterized by an imbalance between bone loss (resorption) and bone formation in favor of bone loss. According to the National Osteoporosis Foundation, half of all Americans over age 50 are expected to have low bone density or osteoporosis by 2020. Osteoporosis is responsible for two million broken bones and $19 billion in related costs every year. Peak bone mass is achieved at around 25-30 years of age. After 45 years of age, bone mass is lost progressively throughout life ultimately leading to age-related osteoporosis. The rate of loss is accelerated for women during the early post-menopausal period, especially at sites with a high component of trabecular bone. The average woman probably has a greater than 40% chance of developing at least one osteoporotic fracture during her lifetime. Osteoporotic fractures, especially of the hip, are associated with a marked reduction in the quality of life and high cost of treatment. According to the Center for Disease Control (CDC) and the National Osteoporosis Foundation, the one year mortality rate for women or men who suffer an osteoporotic hip fracture is on the order of 30%.

Although a range of treatments for osteoporosis have been developed in the past 20 years, there remains an unmet need for one that is not only safe and effective, but also convenient and affordable. Current therapies have significant deficits in one or more of these categories. Here is a summary of the strengths and limitations associated with the principal anti-osteoporotic therapies currently in use.

Antiresorptive agents: Antiresorptive agents prevent osteoclastic bone resorption, usually by directly inhibiting the activity of the cells that resorb bone (osteoclasts) or suppressing their differentiation from precursor cells. These drugs include the bisphosphonates (Alendronate, risedronate, zoledronate, ibandronate), estrogen replacement therapy and RANK Ligand (RANKL) antagonists Denosumab and so on. Anabolic agents include PTH and anti-sclerostin.

At the present time, of these agents, bisphosphonates are the most frequently used anti-osteoporotic treatments. Side effects for all the bisphosphonates (alendronate, ibandronate, risedronate and zoledronate) may include bone, joint or muscle pain. Side effects of the oral tablets may include nausea, difficulty swallowing, heartburn, irritation of the esophagus (tube connecting the throat to the stomach) and gastric ulcer. Inflammation of the eye (called uveitis) is a rare side effect of all bisphosphonates. There have been reports of osteonecrosis (death of bone cells or tissue) of the jaw (ONJ) with bisphosphonate medicines. Recent studies have reported bisphosphonate use (specifically zoledronate and alendronate) as a risk factor for atrial fibrillation in women. The inflammatory response to bisphosphonates or fluctuations in calcium blood levels have been suggested as possible mechanisms. FDA has not yet confirmed a causal relationship between bisphosphonates and atrial fibrillation.

Bisphosphonates treatment for osteoporosis has unique long-term risks. It is now well appreciated, women taking bisphosphonates long-term to for prevent bone loss and osteoporosis are at risk for unusual fractures (“atypical fractures”) in the femur (thigh bone) in the shaft (diaphysis or sub-trochanteric region) of the bone, rather than at the head of the bone, which is the most common site of fracture in osteoporosis. These fractures also appear to be very brittle, as though the bone had become more chalk-like. The current understanding is that long-term remodeling suppression results in over-suppression of bone turnover, which in turn caused bone to accumulate wear and tear micro-cracks. These microcracks eventually unite and propagate, resulting in atypical fractures. Such fractures tend to heal poorly and often require some form of bone stimulation, for example bone grafting as a secondary procedure. Finally, bisphosphonates have extremely long half-lives in vivo (several years) so getting them out of the system to allow bone to recover its tissue health is problematic.

Monoclonal antibody drugs like anti-RANKL antibody that target bone removing cells are very high cost: typically $2,000 or more for a single treatment. These monoclonal antibody drugs must be given every six month by injection and are associated with safety concerns that may increase the risk of skin infection and interfere with other immune reactions. In addition, recent data show that this anti-resorptive strategy leads to the same issue with atypical fractures, as do bisphosphonates, suggesting that it is the long-term resorption suppression without reversibility that is the central problem.

Hormone (Estrogen) Replacement Therapy (HRT) after menopause has also been used. However, findings from the NIH Women's Health Trial with regard to increased risk of increase the risk of blood clot, stroke, and increase in the incidence of breast cancer have effectively removed HRT from the list of viable options to prevent bone loss. Estrogen receptor modulators (SERMs) like raloxifene are still in used but the efficacy at bone loss and fracture prevention is much lower than anti-resorptives like bisphosphonates and denosumab.

Finally the only currently approve anabolic therapy to add back bone to the osteoporotic skeleton is PTH. The approach is problematic. PTH is expensive and requires daily injections. It cannot be used long-term (more than 18 months) due to concerns about bone cancers. Finally, the bone formed by PTH treatment is resorbed once the drug is stopped; the only way to prevent this loss is to given an anti-resportive drug—the problems of which are discussed above. Another anabolic therapy to add back bone to the osteoporotic skeleton is an anti-sclerostin antibody; it is currently under review by the FDA. It is expected to be very high cost: typically $2,000 or more for a single treatment. Furthermore, the bone formed will require an anti-resorptive drug to keep it in place—with the same problems discussed above.

The discussion above is merely provided for general background information and is not intended to be used as an aid in determining the scope of the claimed subject matter.

### BRIEF DESCRIPTION OF THE INVENTION

A method for treating or preventing osteoporosis is provided that treats a patient with methylene blue.

In a first embodiment, a method of providing a therapeutic benefit to a patient is provided. The method comprising steps of: identifying a human patient at risk for osteoporosis, wherein the human patient is at least forty years old; and orally administering a dose of methylene blue to the human patient at least three times per week for at least one year.

In a second embodiment, a method of providing a therapeutic benefit to a patient is provided. The method comprising steps of: diagnosing a human patient as experiencing osteoporosis, wherein the human patient is at least forty years old; and administering a dose of methylene blue to the human patient at least three times per week for at least one year, thereby treating the osteoporosis.

In a third embodiment, a method of providing a therapeutic benefit to a patient is provided. The method comprising steps of: identifying a human patient at risk for osteoporosis, wherein the human patient is at least forty years old; administering methylene blue to the human patient at least three times per week for at least one year; and measuring bone density of the human patient at least once during the at least one year.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic diagram showing the chemical structure of methylene blue and its redox-cycling reaction in vivo; and

FIG. 2A is a MicroCT images from distal femur of estrogen-deficient mice at three months after estrogen depletion by surgical ovariectomy, with and without MB treatment;

FIG. 2B is a graph showing change in bone volumes in the same regions shown in FIG. 2A;

FIG. 3A is a micro-Xray image of aged mouse femur from mouse with no methylene blue treatment;

FIG. 3B is a micro-Xray image of aged mouse femur from mouse after one year duration methylene blue treatment; and

FIG. 4 is a graph depicting porosity of the aged mouse femurs with and without methylene blue treatment.

### DETAILED DESCRIPTION OF THE INVENTION

Disclosed in this specification is a method of using methylene blue (MB) to counteract metabolic oxidative stress as a method to prevent and/or treat osteoporosis. Without wishing to be bound to any particular theory, methylene blue is believed to target the cells (osteocytes) and molecular mechanisms responsible for triggering the remodeling process in the first place. It is believed that targeting the more proximal steps in the development of osteoporosis represents a promising and effective strategy for its prevention and/or treatment. The un-met need of methylene blue as a drug for osteoporosis is because methylene blue is able to maintain normal physiological pathways for bone formation and resorption to maintain bone quality.

Osteoporosis is a condition associated with significant accumulation of oxidative stress in the orchestrator cell (osteocytes) leading to cell death that triggers the downstream events initiating bone loss. Current medications effectively reduce fracture risk and increase BMD by blocking bone resorption (even when is required to prevent microcrack accumulation) or triggering abnormal bone formation but also associated with diverse side effects as described above. Metabolic oxidative stress is developed when there is malfunctioned mitochondria and alteration in cellular energy supply. Methylene blue has the ability to counteract mitochondria function and maintain ATP levels in the cell as needed.

Since it was first synthesized in 1876, methylene blue has been used for a variety of medicinal purposes. For example, it has been reported that bipolar manic-depressive patients treated with 300 mg per day methylene blue for one year were significantly less depressed than when treated with placebo. As another example, methylene blue at dosages of 65 mg taken three times a day has been reported to be useful in the management of chronic renal calculus disease. It has also been reported that methylene blue, injected at a dose of 1 mg per kg, improved brain oxidative metabolism and memory retention in rats, and that senescence-enhanced oxidative stress is associated with deficiency of mitochondrial cytochrome c oxidase in vascular endothelial cells.

Methylene blue has a number of advantages as a drug to prevent oxidative stress in bone cells and in turn prevent bone loss: (1) methylene blue is approved by the U.S. Food and Drug Administration (FDA) approved for topical, injection and oral administration. Current indications for methylene blue that are approved by the FDA are hereditary and also acquired methemoglobinemias, short term treatment of elderly patients to prevent urinary tract infections, and intraoperative visualization of nerves, nerve tissues, and endocrine glands as well as of pathologic fistulae are among the major uses (2) methylene blue is a potent antioxidant which readily accumulates in bone (3) MB is an artificial electron acceptor/donor in mitochondria and therefore supports ATP synthesis (4) methylene blue improves downregulation of pro-apoptotic pathways (apoptosis is programmed cell death), and support cell survivor and renewal of ATP. Bone cell apoptosis in response to estrogen loss and disuse has been shown to be a major trigger for osteoporosis (5) methylene blue has an excellent safety profile and is effective in such low doses that toxicity is highly unlikely (6) methylene blue is to believe confers benefits in healthy subjects as well (7) methylene blue is short-acting, which allows for the development of a meaningful on-off treatment strategy to prevent bone loss and also conserve the mechanical properties of healthy bone and (8) methylene blue has a low cost.

Methylene blue has been found to protect bone cells from oxidative stress and prevent cancellous bone loss after estrogen removal in mice model at effective concentrations that are orders of magnitude lower than previously reported therapeutic doses of methylene blue. Using the same animal model, methylene blue was able to prevent osteocyte apoptosis the trigger of bone remodeling and also maintained bone mechanical property and bone mineral density. Methylene blue treatment was found to prevent oxidative stress formation and accumulation in bone cells which helps maintain cell viability and activity and prevent the development of osteoporosis.

Methylene blue serves as a redox compound that at low doses (1-5 mg per kg) improves mitochondrial respiration and prevents free radical damage. Low-dose methylene blue is believed to act on the electron transport chain and increases cellular oxygen consumption by a mechanism of action that involves accepting electrons from molecular oxygen. Methylene blue treatment maintains healthy bone and might promote bone formation in already osteoporotic patient. Additionally, methylene blue may be used to delay the onset of aging related disease that are initiated with increase in metabolic oxidative stress.

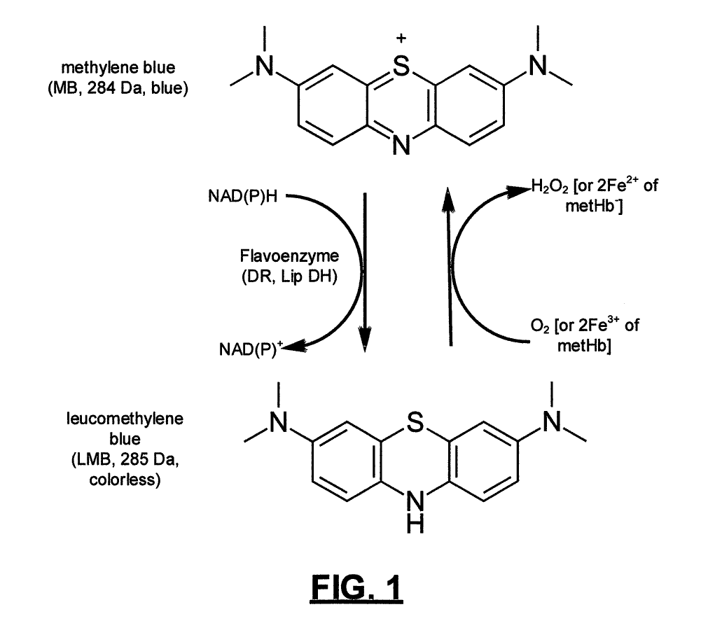

FIG. 1 depicts the chemical structure of methylene blue. Methylene blue is a redox-cycling phenothiazine drug in vivo. Methylene blue (MB) is spontaneously or enzymatically reduced by NADPH and the resulting uncolored leucoMB is reoxidized by molecular oxygen (O₂) or by iron(III)-containing compounds like methemoglobin. Precious NADPH and O₂ are wasted and hydrogen peroxide is produced in each round of the cycle. There are also pharmacologic activities of MB which do not depend on its redox properties.

### Example 1 Methylene Blue Bone Loss Prevention Experiments

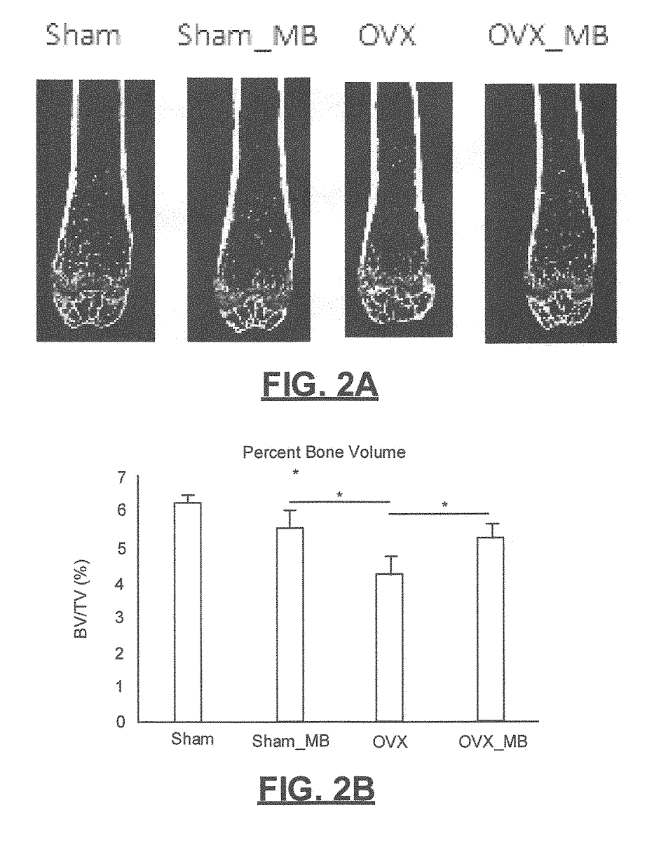

Young adult mice were subjected to surgical ovariectomy (OVX) to deplete estrogen. This is widely accepted model for inducing post-menopausal type bone loss. It is the standard used by pharmaceutical companies in preclinical trials and is required by the FDA to confirm efficacy of an agent in preventing bone loss. Seven sham control mice received a surgical incision and wound closure but ovaries were left intact. Mice were return to their cages after surgery. Six “MB” mice received methylene blue in the cage water (0.2% in cage water supply starting 24 hours before surgery) which they were allowed to consume ad libitum for the duration of the study (three months); the remainder of the mice received normal cage water. Mice were sacrificed at three months post-OVX and bones were examined using microCT. The results are depicted in FIG. 2A and FIG. 2B which show bone changes with estrogen loss (OVX) were prevented by systemic treatment with methylene blue (MB). FIG. 2B is a graph of Bone Volume over Total Volume (BV/TV) for the groups of mice. OVX without methylene blue treatment resulted in approximately 40% reduction in bone volume. This bone loss was almost completely prevented in mice receiving methylene blue.

### Example 2

Example 1 showed that methylene blue (MB) prevents the post-menopausal (Type 1) osteoporosis that occurs with estrogen loss, typical of bone loss in women after menopause. This was done using surgical removal of the ovaries from adult mice, which is the standard preclinical animal model for post-menopausal osteoporosis.

Example 2 presents conclusive evidence showing that methylene blue will also prevent Type 2 osteoporosis, the age-related bone loss that occurs in both men and women with aging. This loss starts at about 50 year of age in humans.

A one year old mouse is biologically similar to a 45-50 year-old human. Starting at 1 year of age, methylene blue was administered in the cage water supply (0.2% by weight) for one year to three male mice. Control mice received normal water. Animals were free to consume water and food ad libitum. Bones were examined when the mice were two years old, which is similar to a 75-80 year-old person.

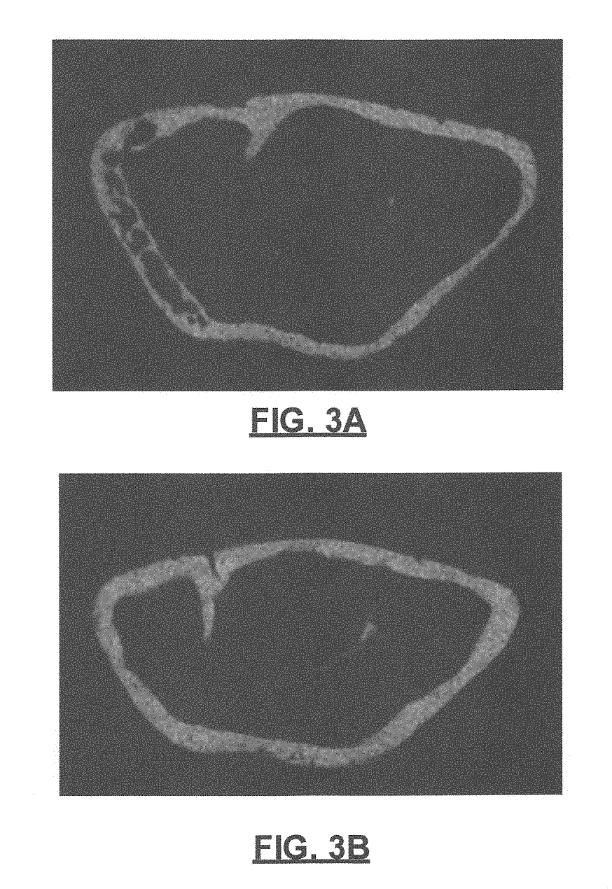

FIG. 3A is a micro-Xray image of aged mouse femur from mouse with no treatment. Large holes or pores are clearly seen in the bone, as is thinning of the bone cortex. This is normal age-related osteoporosis.

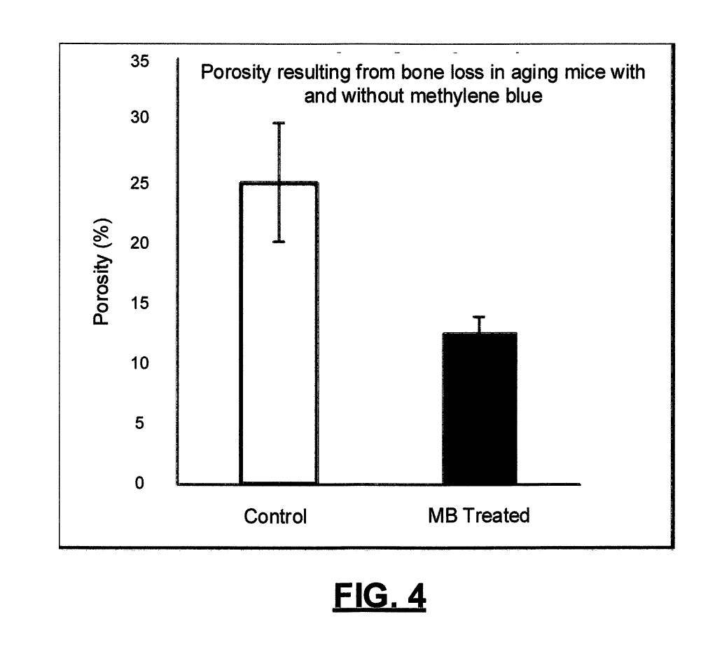

FIG. 3B is a micro-Xray image of aged mouse femur from mouse after one year duration methylene blue treatment. No increases in porosity or cortex thinning are seen. Methylene blue treatment prevented Age related osteoporosis from developing. Methylene blue treatment dramatically reduced bone loss. It prevented aging bone from becoming porous and also prevented the cortex or shell of the bone from thinning—which are the hallmark features of age related osteoporosis. These differences are demonstrated clearly in the micro-Xray images shown in FIG. 3A and FIG. 3B. FIG. 4 quantifies these differences in a graph of bone porosity which showed bones treated with methylene blue had about half the porosity compared to bones from control mice.

In one embodiment, methylene blue is administered to a patient over a prolonged period of time to prevent or treat osteoporosis. For example, a dose of methylene blue may be administered at least three times per week for at least one year. In one embodiment, the methylene blue is administered orally. In another embodiment, the dose is administered daily for at least one year. In another embodiment, the dose is administered for a certain treatment period which is followed by a non-treatment period (e.g. six months of treatment and six months of non-treatment or three months of treatment and three months of non-treatment followed by repeating these treatment/non-treatment cycles). In both of these embodiments, at least half of the year is spent undergoing methylene blue treatment (i.e. at least 180 treatment days over a given year). The dosage of methylene blue is at least 1 mg methylene blue per kg body weight of the patient. Because age-related osteoporosis does not occur under the age of forty (or even fifty for many patients) the patient is over forty years old or, in another embodiment, over fifty year old. In one embodiment, the bone density of the patient is measured to determine the effectiveness of the treatment. For example, the bone density of the patient may be measured at least once during the year when methylene blue is administered. In one such embodiment, the bone density is measured in the latter half of the treatment time period (e.g. after at least six months of methylene blue treatment).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of providing a therapeutic benefit to a patient, the method comprising steps of:
identifying a human patient at risk for osteoporosis, wherein the human patient is at least forty years old; and
administering a dose of methylene blue to the human patient at least three times per week for at least one year.

2. The method as recited in claim 1, wherein the step of administering administers the dose daily for the at least one year.

3. The method as recited in claim 1, wherein the step of administering administers the dose at least 180 times over the at least one year.

4. The method as recited in claim 1, wherein the dose of the methylene blue is administered at a dosage of at least 1 mg methylene blue per kg body weight of the human patient.

5. The method as recited in claim 1, wherein the human patient is at least fifty years old.

6. A method of providing a therapeutic benefit to a patient, the method comprising steps of:
diagnosing a human patient as experiencing osteoporosis, wherein the human patient is at least forty years old; and
administering a dose of methylene blue to the human patient at least three times per week for at least one year, thereby treating the osteoporosis.

7. The method as recited in claim 6, wherein the step of administering administers the dose daily for the at least one year.

8. The method as recited in claim 6, wherein the step of administering administers the dose at least 180 times over the at least one year.

9. The method as recited in claim 6, wherein the dose of the methylene blue is administered at a dosage of at least 1 mg methylene blue per kg body weight of the human patient.

10. The method as recited in claim 6, wherein the human patient is at least fifty years old.

11. A method of providing a therapeutic benefit to a patient, the method comprising steps of:
identifying a human patient at risk for osteoporosis, wherein the human patient is at least forty years old;
administering methylene blue to the human patient at least three times per week for at least one year; and
measuring bone density of the human patient at least once during the at least one year.

12. The method as recited in claim 11, wherein the step of measuring is performed after at least six months of the administering methylene blue to the human patient.

13. The method as recited in claim 12, wherein the step of administering administers the dose daily for the at least one year.

14. The method as recited in claim 12, wherein the step of administering administers the dose at least 180 times over the at least one year.

15. The method as recited in claim 12, wherein the human patient has been diagnosed as experiencing osteoporosis.

16. The method as recited in claim 12, wherein the step of administering methylene blue administers the methylene blue orally.
